(19) United States
(12) Patent Application Publication
(10) Pub. No.: US 2020/0148998 A1
(43) Pub. Date: May 14, 2020

(54) Method For Production of an Engineered Liquid

(71) Applicants: Faramaz Fred Faramarzi, Encino, CA (US); Sabrina Faramarzi, Encino, CA (US)

(72) Inventors: Faramaz Fred Faramarzi, Encino, CA (US); Sabrina Faramarzi, Encino, CA (US)

(21) Appl. No.: 16/531,052

(22) Filed: Aug. 3, 2019

Related U.S. Application Data
(60) Provisional application No. 62/760,491, filed on Nov. 13, 2018.

(51) Int. Cl. C12N 1/20; C12N 1/12

(52) U.S. Cl. CPC C12N 1/20; C12N 1/12

(57) ABSTRACT

A method for the production of an engineered liquid for use in a system to duplicate a flammable gas. The engineered liquid facilitates duplicating a quantity of almost any selected or desired flammable gas. The method comprises the following steps: place a bacteria into a container; add an algae into the container; add water to the bacteria and algae to create a mutated bacteria solution; add a quantity of bone powder; add a quantity of nutrients and water; and allow the mutated bacteria solution to culture.

### TECHNICAL FIELD

The invention generally pertains to the method of production of an engineered liquid, and more particularly to a method for the production of an engineered liquid that is capable of duplicating a flammable gas.

### BACKGROUND OF THE INVENTION

Flammable gasses are gasses that burn in the presence of an oxidant when provided with a source of ignition. The risk of ignition increases in relation to the amount of gas present. If the concentration of a flammable gas in an environment exceeds the upper explosive limit (UEL), the environment becomes ‘too rich to burn’, which creates the opposite effect, instead reducing a chance of ignition.

Flammable gasses can be produced both naturally and artificially. Natural gases, like Biogas or Methane, are generally created from decaying organic matter. These same chemicals can also be created in a lab setting through a chemical process.

Both the natural and artificial method of natural gas production have drawbacks, including but not limited to: maintenance; monitoring; strong, pungent odors; dangerous bacteria; decaying matter; byproducts; issues with capturing the gasses; issues with storing the gasses; the lengthy process for producing, capturing, storing, purifying, and overall creating a useable natural gas product; and cost.

Gas and energy companies developed an infrastructure based on the two most common forms of flammable gasses: natural gas and coal based gas, which is what the energy industry currently runs on. The majority of natural gas is Methane (CH₄), which is also a byproduct of petroleum and other fossil fuels. However, Methane is rarely found in a solely pure form in nature, usually found in conjunction with other hydrocarbons including, but not limited to: Butane, Ethane, and Propane. Manufactured coal gas, and its several variants, was commonly used in the 19th century and beyond. The simpler process consists of heating coal, or other similar organic substances, to produce a flammable gas. The resulting gas is a combination of carbon monoxide (CO), hydrogen (H) and other gasses depending upon the exact process and staring substances. This method has long been utilised due to the comparative ease of procuring coal and therefore producing coal based gas. These gases have long proven to be consistent fuel sources for residential, commercial, and industrial energy needs and endeavors.

The discovery of massive natural gas fields in Southwestern America, in conjunction with technological advancements in long distance pipeline construction, dramatically altered the gas industry moving into the twentieth century. The sheer volume of gas fields emphasized a need for advancements in the natural gas infrastructure, materials, purification, and technology fields.

In the time period surrounding World War II, a second era of rapid gas industry growth occurred, with a national market for national gas consumption emerging. During the last half of the twentieth century, natural gas consumption in the United States ranged from about 20-30% of total national energy utilization.

The natural gas industry is not without its issues. First, there are periodic shortages of natural gas due to a variety of factors, ranging from regulatory issues to OPEC oil embargos to increased general needs. While it is the cleanest burning of all fossil fuels, natural gas exists in limited supply. Estimates of future natural gas availability vary widely, from mere hundreds to several thousands of years. Such estimates are dependent upon the technology that must be developed to produce natural gas. Currently, natural gas is the most economically efficient source of energy used throughout the world. A multitude of natural and flammable gases are used annually to power homes, factories and automobiles. These natural gases are also used in power plants to produce electricity and other forms of power.

Second, the extraction methods for obtaining these flammable gasses have an impact on the environment. There are several common places to extract and obtain flammable gases: coal bed methane (CBM), gas shale, natural gas from oil wells from sand (tight gas), gas hydrates (clathrates), fossil fuels, and extraction of fuel from CO₂. In addition, there are plant-based technologies, such as from corn. Many others are in the research and development stage. Presently, all of the available methods and sources have a high cost of production and present extreme challenges. These include reducing damage to the environment, and reducing the cost of extraction, transportation and logistical strategies to get the fuel to the final destination.

From start to finish, the process of obtaining natural gas is extensive and invasive. For example, to capture hydrocarbon gas from a natural gas reserve initially requires deep drilling to find the reservoir, capturing the gas as it is expelled from the underground pocket, and transferring the crude gas to a refinery. At that refinery, the gas is separated into its parts (for example: propane, methane, butane, and a bevy of other chemicals or substances may all be present in the sample obtained when only methane is wanted or needed). The separated gases are then individually stored and transferred to customer either by gas tankers or through built in gas line infrastructure, which are both relatively costly methods. Further, those separated gases are not all necessarily needed; some are too harmful to use, some are scarce, some are overproduced, and some are entirely unnecessary.

These final product natural gas fuels and natural-gas condensate consist of several hydrocarbon gases. The most common condensates consist of a combination of: butane; cyclobutene; cycloheptane; cyclohexane; cyclopentane; cyclopropane; dimethylpropane; ethane; Ethylene; heptane; hexane; methane; methyl butane; methanol, methyl propane; pentane; propane, and/or other hydrocarbon flammable gases.

In general, the major costs associated with natural gas production do not originate from the initial drilling and securing of the gas but instead with the transfer, refinement, and storage of the gas. These costs are relayed to the final consumer and prove a major burden. The necessity for storage, transportation, and infrastructure is a multi-billion dollar industry.

On some oil and natural gas drilling sites, natural gases are simply left to burn when they are not desired, which causes other environmental issues as well as incurring additional economical concerns, as related to cleanup.

The instant invention offers a solution to these problems by creating an engineered liquid that is capable of duplicating the properties of a hydrocarbon flammable gas.

### PRIOR ART

A search of the prior art did not disclose any literature or patents that read directly on the claims of the instant invention. For background purposes and indicative of the art to which the invention relates, reference may be made to the following remaining patents found in the patent search. However, the following U.S. patents are considered related:

| PATENT NO. | INVENTOR | ISSUED |
|---|---|---|
| 4,869,894 | Wang, et al | Sep. 26, 1989 |
| 5,229,089 | Ramachandran, et al | Jul. 20, 1993 |
| 2008/0257719 | Suratt | Oct. 23, 2008 |
| 2,822,888 | MacLaren | Feb. 11, 1958 |
| 3,150,942 | Srini | Sep. 29, 1984 |
| 4,077,779 | Sircar, et al | Mar. 7, 1978 |
| 4,553,981 | Fuderer | Nov. 19, 1985 |
| 4,963,339 | Krishnamurthy, et al | Oct. 16, 1990 |

### BRIEF DESCRIPTION OF THE DRAWINGS

FIG. 1 shows a table with the ratios necessary to engineer one thousand (1000) liters of an engineered liquid.

### DISCLOSURE OF THE INVENTION

In view of the above disclosure, the primary object of the invention is to provide a method for the production of an engineered liquid that is capable of duplication of a flammable gas.

In addition to the primary object, it is also an object of the invention to provide an engineered liquid that:
allows flammable gas producers to save a range of forty to eighty percent at final production cost,
can be utilized in any city in the world, on any scale,
allows one preselected gas to be produced with the exact kind of gases independently and at a destination location,
allows a customer to consecutively produce multiple flammable gases,
reduces refinery costs along with the associated refinery process waiting period,
allows gas companies to extensively reduce need to transport gas through expensive pipelines, by ship, or by truck,
allows storage needs to be lessened or eliminated,
extensively reduces the need for drilling, capturing gases, and risk of explosion,
extensively allows less drilling to be required and less unwanted gases that are released into the environment, lessening environmental pollution,
produces only the desired gas, unlike a conventional natural gas refinery process in which many unwanted or hazardous gases are separated as part of the process,
allows the life expectancy of available natural gases to be increased by up to four times.

These and other objects and advantages of the present invention will become apparent from the subsequent detailed description of the preferred embodiment and the appended claims taken in conjunction with the accompanying drawings.

### SUMMARY OF THE INVENTION

A method for the production of an engineered liquid for use in a system to duplicate a flammable gas is disclosed. The engineered liquid facilitates duplicating a quantity of almost any selected or desired flammable gas.

The method comprises the following steps: place bacteria into a container; add an algae into the container; add water to the bacteria and algae to create a mutated bacteria solution; add a quantity of bone powder; add a quantity of nutrients and water; and allow the mutated bacteria solution to culture.

The bacteria and algae are originally in a dormant state; are derived from organic matter; and are a powder. The water added is room temperature and the mutated bacteria solution is created by adding water to the bacteria and the algae in the container to activate the bacteria and algae to react with each other. The mutated bacteria solution is stored at a temperature ranging from 5°-45° Celsius and is non-flammable.

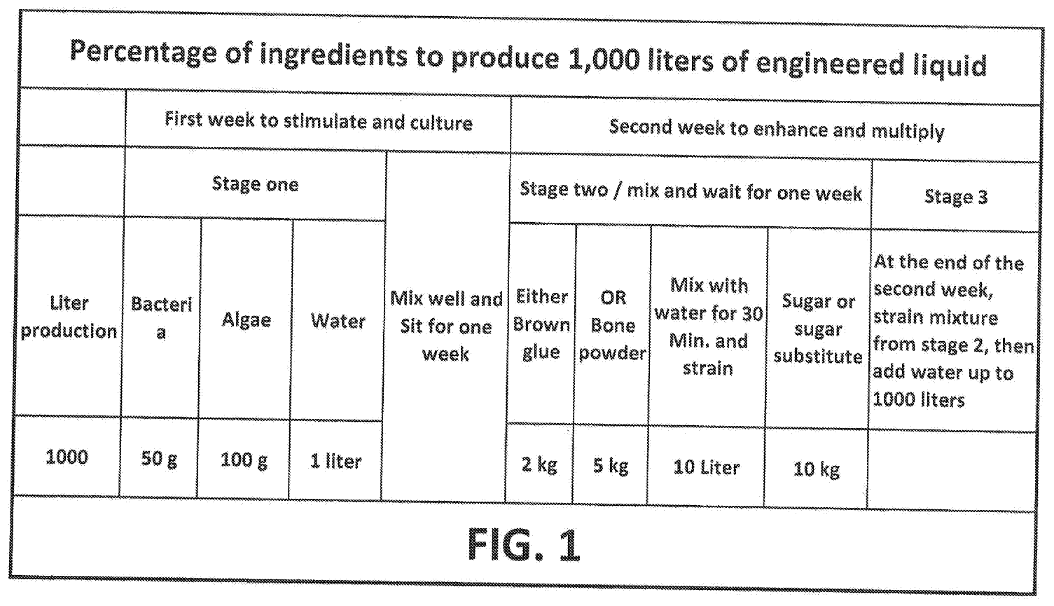

The amount of the bacteria added is according to the formula in FIG. 1. The bacteria and algae are dormant; and the algae is derived from green algae, red algae, cyanobacteria, and other edible forms of algae; and is a powder. The amount of the algae added is according to the formula in FIG. 1. The water is added to increase the amount of the engineered liquid to a desired volume and the amount of water added is according to the formula in FIG. 1. The bone powder is produced by heating animal by-products to their boiling point then grinding the resulting in a fine powder or the bone powder is produced by heating animal bones to their boiling point then grinding the resulting mixture to a fine powder. The bone powder is a powder or a liquid, or is gelatinous. The nutrients consist of glucose, glucose derived substances, sugar, sugar derived substances, or organic by-products or materials. The amount of water added to the mutated bacteria solution is according to the formula in FIG. 1. The engineered liquid solution is capable of replicating and producing a flammable gas and is first introduced to a flammable gas, then introduced to Deoxygenated or Ambient Air or Nitrogen Gas (DAANG) to extract, carry out, and transfer the flammable gas from the engineered liquid.

The method for producing one thousand (1000) liters of an engineered liquid, comprising the following steps: Stage one: add fifty (50) to one hundred (100) grams of bacteria to a container; add one hundred (100) to two hundred (200) grams of algae to the container; add one (1) liter of water to the container; mix the bacteria, the algae, and the water; allow the to sit for one week to create a mutated bacteria; Stage two: add five (5) kilograms of bone powder or two (2) kilograms of brown glue mixed with ten (10) liters of water to the mutated bacteria; add ten (10) kilograms of sugar; allow the to sit for one week to create the engineered liquid; Stage 3: strain the engineered liquid for purity, keeping the liquid; and add water, as needed, to reach one thousand (1000) liters.

The bacteria and algae are originally in dormant states; are derived from organic matter; and are powders. The mutated bacteria solution is stored at a temperature ranging from 5°-45° Celsius. Water is added to increase the amount of the engineered liquid to the desired volume. The engineered liquid is also stored at a temperature ranging from 5°-45° Celsius and is non-flammable.

The method of introduction of DAANG is by passing the flammable gas through the engineered liquid. The flammable gas is from the hydrocarbon family of flammable gasses or other flammable gasses. The algae is derived from green algae, red algae, cyanobacteria, and other edible forms of algae. The water is purified by boiling then cooling the liquid. Five (5) kilograms of bone powder can be replaced with two (2) kilograms of bone glue; and the bone powder is produced by heating animal by-products to their boiling point then grinding the resulting to a fine powder or by heating animal bones to their boiling point then grinding the resulting to a fine powder. The bone powder is a powder or a liquid or a gelatinous; and the sugar is sugar, a sugar derived substance, glucose, or glucose derived substance or an organic by-products or material. The engineered liquid solution is capable of replicating and producing a flammable gas. The engineered liquid is first introduced to a flammable gas, then introduced to DAANG to extract, carry out, and transfer the flammable gas from the engineered liquid. The method of introduction of DAANG is by passing the flammable gas through the engineered liquid.

In a particular embodiment, in less than one hour, a single unit of any flammable or hydrocarbon gas will yield up to at least double the quantity of the same gas back. DAANG in combination with the engineered liquid and a desired hydrocarbon flammable gas within a specially designed container will result in production of at least two times more of the same originally introduced hydrocarbon gas and the increased volume of flammable gas.

While the invention has been described in detail and pictorially shown in the accompanying FIG. it is not to be limited to such details, since many changes and modifications may be made to the invention without departing from the spirit and the scope thereof. Hence, it is described to cover any and all modifications and forms that may come within the language and scope of the claims.

1. A method for producing an engineered liquid, the method comprising:
place bacteria into a container;
add algae into the container;
add water to the bacteria and algae mixture to create a mutated bacteria solution;
add a quantity of bone powder;
add a quantity of nutrients and water; and
allow the mutated bacteria solution to culture.

2. The method for producing an engineered liquid in claim 1, wherein the bacteria is originally in a dormant state.

3. The method for producing an engineered liquid in claim 1, wherein the bacteria is derived from organic matter.

4. The method for producing an engineered liquid in claim 1, wherein the bacteria is a powder.

5. The method for producing an engineered liquid in claim 1, wherein the water added is room temperature.

6. The method for producing an engineered liquid in claim 1, wherein the mutated bacteria solution is created by adding water to the bacteria and the algae in the container to activate the bacteria and algae mixture to react with each other.

7. The method for producing an engineered liquid in claim 1, wherein the mutated bacteria solution is stored at a temperature ranging from 5°-45° Celsius.

8. The method for producing an engineered liquid in claim 1, wherein the engineered liquid is non-flammable.

9. A method for producing one thousand (1000) liters of an engineered liquid comprising:
Stage one comprises:
add fifty (50) to one hundred (100) grams of bacteria to a container;
add one hundred (100) to two hundred (200) grams of algae to the container;
add one (1) liter of water to the container;
mix the bacteria, the algae, and the water;
allow the mixture to sit for three (3) to ten (10) days to create a mutated bacteria;
Stage two comprises:
add five (5) kilograms of bone powder or two (2) kilograms of brown glue mixed with ten (10) liters of water to the mutated bacteria;
add ten (10) kilograms of sugar;
allow the mixture to sit for seven (7) to ten (10) days to create the engineered liquid;
Stage 3:
strain the engineered liquid for purity, keeping the liquid; and
add water, as needed, to reach one thousand (1000) liters.

10. The method for producing the engineered liquid from claim 9, wherein the bacteria and algae are originally in a dormant state.

11. The method for producing the engineered liquid from claim 9, wherein the bacteria is derived from organic matter.

12. The method for producing the engineered liquid from claim 9, wherein the bacteria and the algae are a powder.

13. The method for producing an engineered liquid in claim 9, wherein the mutated bacteria solution is stored at a temperature ranging from 5°-45° Celsius.

14. The method for producing the engineered liquid from claim 9, wherein the water is added to increase the amount of the engineered liquid to the desired volume.

15. The method for producing an engineered liquid in claim 9, wherein the engineered liquid is stored at a temperature ranging from 5°-45° Celsius.

16. The method for producing the engineered liquid from claim 9, wherein the engineered liquid is non-flammable.
